(19) United States
(12) Patent Application Publication
(10) Pub. No.: US 2024/0285512 A1
(43) Pub. Date: Aug. 29, 2024

(54) ORAL CARE COMPOSITIONS

(71) Applicant: Firmenich SA, Satigny (CH)

(72) Inventors: Yang HUANG, Shanghai (CN); Monica BANDERA, Satigny (CH); Jan HAINES, Singapore (SG); Brooke Xiao-Xi LIN, Shanghai (CN); Qingbo OUYANG, Shanghai (CN)

(21) Appl. No.: 18/573,258

(22) PCT Filed: Jun. 21, 2022
(86) PCT No.: PCT/EP22/66805

§ 371 Date: Dec. 21, 2023

(30) Foreign Application Priority Data
Jun. 24, 2021 (WO) PCT/CN2021/102153
Aug. 4, 2021 (EP) 21189678.2

(51) Int. Cl. A61K 8/9789; A61K 8/9794; A61Q 11/00; C12Q 1/18

(52) U.S. Cl. CPC A61K 8/9789; A61K 8/9794; A61Q 11/00; C12Q 1/18

(57) ABSTRACT

Described herein is an oral care composition including one or more of the following compounds or compositions: myristic acid, cis-3-hexenylhexanoate, citronellol, nootkatone, ambrette seed absolute, spinach absolute, helichrysum italicum absolute, butanol, geraniol, beta ionone, isoamyl alcohol, methyl (2-pentyl-3-oxocyclopentyl) acetate, anethole, decanoic acid, undecanoic acid, nutmeg oil, clary sage oil, lemongrass oil, lime oil, peppermint oil (for example, mint piperita reco nat, mint piperita boulder sx, or mint piperita yakima single cut), gamma octalactone, 3-hexenyl acetate, lavender oil, cinnamaldehyde, clove oil, mint arvensis oil (for example mint arvensis tpc), linseed oil, and thymol. Also described herein are oral care products incorporating such compositions and related methods.

### INTRODUCTION

Oral diseases such as tooth decay are very prevalent in society. They can cause chronic and acute pain to sufferers and can be fatal. Oral hygiene plays an important role to mitigate the effects of such diseases. Examples of oral hygiene include regular tooth brushing, flossing, tongue scrapers and of course routine visits to an oral healthcare practitioner.

Oral care preparations, such as, for example, mouthwashes or mouth rinses have been known for a long time. They are developed to clean and refresh the oral cavity or oral surface by inhibiting or killing the microorganisms that cause malodor, dental caries, tooth decay, gum diseases, gingivitis, and periodontal disorders.

While there are known oral care compositions which have antimicrobial activity, there is a need in society for additional compositions to improve oral hygiene and reduce the instances of oral diseases.

### SUMMARY OF THE INVENTION

The present invention provides a solution to the above mentioned problem by providing an oral care composition comprising one or more of the following compounds or compositions: myristic acid, cis-3-hexenylhexanoate, citronellol, nootkatone, ambrette seed absolute, spinach absolute, helichrysum italicum absolute, butanol, geraniol, beta ionone, isoamyl alcohol, methly (2-pentyl-3-oxocyclopentyl) acetate, anethole, decanoic acid, undecanoic acid, nutmeg oil, clary sage oil, lemongrass oil, lime oil, peppermint oil (for example, mint piperita reco nat, mint piperita boulder sx, or mint piperita yakima single cut), gamma octalactone, 3-hexenyl acetate, lavender oil, cinnamaldehyde, clove oil, mint arvensis oil (for example mint arvensis tpc), linseed oil, and thymol.

An embodiment of the invention provides an oral care composition of the invention is wherein the compounds or compositions is selected from: myristic acid, cis-3-hexenylhexanoate, citronellol, nootkatone, ambrette seed absolute, spinach absolute, helichrysum italicum absolute, butanol, geraniol, beta ionone, isoamyl alcohol, methly (2-pentyl-3-oxocyclopentyl) acetate, nutmeg oil, clary sage oil, lemongrass oil, peppermint oi and gamma octalactone.

An embodiment of the invention provides an oral care composition of the invention is wherein the compounds or compositions is selected from: myristic acid, citronellol, nookatone, nutmeg oil, clary sage oil, lemongrass oil, peppermint oil and gamma octalactone.

An embodiment of the invention provides an oral care composition of the invention is wherein the compounds or compositions is at a concentration of from 0.001 to 5%, particularly preferably from 0.005 to 3%, preferably 0.05 to 2.5%, preferably 0.1 to 2.5%, preferably 0.5 to 2% in each case with respect to the total weight of the composition.

An embodiment of the invention provides an oral care composition of the invention is wherein said composition further comprising one or more of the following ingredients: flavor system, cooling agents, surfactants, humectants, pigments, antibacterial agents, thickening agents, fluoride sources, tartar control agents and/or preservatives.

An embodiment of the invention provides an oral care composition of the invention is wherein the composition is coated or encapsulated.

A further aspect of the invention provides an oral care product or product for nutrition or pleasure comprising an oral care composition of the invention.

An embodiment of the invention is wherein the oral care product is selected from the group comprising of tooth paste, tooth powder, tooth gel, tooth cleaning liquid, tooth cleaning foam, tooth tablets, mouth wash, mouth rinse, mouth spray, dental floss, chewing gum, dental aligners, lozenges, dentures effervescent tablets; dentures effervescent tablets and dental aligners.

A further aspect of the invention provides a method of treatment or prevention of erosive tooth demineralization, gingivitis, plaque, periodontitis, halitosis and/or dental caries, the method comprising the application to the oral cavity of a person in need thereof a composition according to any preceding claim.

A further aspect of the invention provides the use of the oral care composition of the invention for the treatment or prevention of erosive tooth demineralization, gingivitis, plaque, periodontitis, halitosis and/or dental caries.

A further aspect of the invention provides a method of identifying a test composition having use in oral healthcare comprising
(a) assaying whether the test composition reduces bacteria adhesion, and/or,
(b) assaying whether the test composition reduces biofilm formation, and/or,
(c) assaying whether the test composition disrupts the biofilm,
wherein a composition which is positive in one or more assays is of use in an oral healthcare composition.

An embodiment of the method of identifying a test composition is wherein the assay of step (a) comprises exposing a substrate to saliva and/or bacteria in the addition or absence of a test composition and determining whether the test composition reduces bacteria adhesion to the substrate.

An embodiment of the method of identifying a test composition is wherein the assay of step (b) comprises allowing exposing a substrate to saliva and/or bacteria under culture conditions for at least 24 hours then exposing the cultured mixture to a test composition and determining whether the test composition reduces biofilm formation to the substrate.

An embodiment of the method of identifying a test composition is wherein the assay of step (c) comprises allowing a biofilm to form on a substrate by exposing a substrate to saliva and/or bacteria under culture conditions for at least 72 hours then exposing the cultured mixture to a test composition and determining whether the test composition disrupts the biofilm.

An embodiment of the method of identifying a test composition is wherein the effect of the test composition on the biofilm is determined by harvesting the biofilm and determining the amount and type of bacteria present in the biofilm.

An embodiment of the method of identifying a test composition further comprises preparing an oral care composition using a test composition which is positive in one or more assays.

### DESCRIPTION OF THE FIGURES

FIG. 1. Change of plaque index between sampling time points and bacterial load in used mouth rinse on Day 7.

### DESCRIPTION OF THE INVENTION

Oral biofilm is a term well known in the art. It refers to a community of micro-organisms and their products embedded in a matrix which adheres to tooth enamel and to soft tissues in the mouth including the tongue. The cooperative nature of the microbial community within the biofilm provides advantages to the participating bacteria, from a broader habitat range enhancing growth to greater resistance to host defenses and antimicrobial agents that also enhance the microbial communities' pathogenicity.

While oral biofilm is a commonly found in well-kept mouths, in some situations the biofim can cause oral pathologies, including erosive tooth demineralization, gingivitis, plaque, periodontitis, halitosis and/or dental caries.

Although mechanical disturbance of the biofilm through teeth brushing or flossing can remove some of the pathogenic microorganisms and aid oral hygiene, the biofilm can rapidly regenerate. Hence there is a need to identify compounds and compositions which can be used either alone or in combination with mechanical disruption to retard the development of oral biofilm.

The present inventors sought to identify compounds or compositions which can be used in oral care compositions to improve oral hygiene.

They devised a method of identifying whether a test compound or composition can have utility in an oral care composition by: (a) assaying whether the test composition reduces bacteria adhesion, and/or, (b) assaying whether the test composition reduces biofilm formation, and/or, (c) assaying whether the test composition disrupts the biofilm, wherein a composition which is positive in one or more assays is of use in an oral healthcare composition.

Further information concerning the performance of the assays can be identified in the accompanying examples and further described below.

From this work the inventors identified a number of compounds or compositions which modulate bacteria adhesion, and/or biofilm formation, and/or disrupts the biofilm: myristic acid, cis-3-hexenylhexanoate, citronellol, nootkatone, ambrette seed absolute, spinach absolute, helichrysum italicum absolute, butanol, geraniol, beta ionone, isoamyl alcohol, methly (2-pentyl-3-oxocyclopentyl) acetate, anethole, decanoic acid, undecanoic acid, nutmeg oil, clary sage oil, lemongrass oil, lime oil, peppermint oil (for example, mint piperita reco nat, mint piperita boulder sx, or mint piperita yakima single cut), gamma octalactone, 3-hexenyl acetate, lavender oil, cinnamaldehyde, clove oil, mint arvensis oil (for example mint arvensis tpc), linseed oil, and thymol.

Hence the invention provides oral care compositions comprising one or more of the compounds or compositions as mentioned herein which have anti-oral biofilm effects, i.e. they reduce bacteria adhesion, and/or reduce biofilm formation, and/or disrupt the biofilm. They hence have utility in preventing or treating oral pathologies, including erosive tooth demineralization, gingivitis, plaque, periodontitis, halitosis and/or dental caries.

By “reduces” we include where the compounds or composition have a positive result in one or more of the assays used in the aspects of the invention described herein. By “positive result” the inventors mean that the composition gives a statistically significant effect, that effect being a reduction, in an assay compared to a control reaction which includes the same sample mixture but lacking the test compound or composition.

As used herein, a “statistically significant effect” is where a compound or composition has a p value lower than 0.05 (i.e. a confidence interval of 95%) as measured in a Student's T-test. As can be appreciated such a reduction would be beneficial in reducing the likelihood of a subject developing an oral pathology, as listed above.

An embodiment of the invention is wherein the oral care product is selected from the group comprising of tooth paste, tooth powder, tooth gel, tooth cleaning liquid, tooth cleaning foam, tooth tablets, mouth wash, mouth rinse, mouth spray, dental floss, chewing gum, dental aligners, lozenges, dentures effervescent tablets; dentures effervescent tablets and dental aligners.

As used herein, the term “oral healthcare composition” refers to a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral healthcare composition may be in various forms including tooth paste, tooth powder, tooth gel, tooth cleaning liquid, tooth cleaning foam, tooth tablets, mouth wash, mouth rinse, mouth spray, dental floss, chewing gum, dental aligners, lozenges, dentures effervescent tablets; dentures effervescent tablets and dental aligners.

The oral healthcare composition may also be incorporated onto floss, strips or films for direct application or attachment to oral surfaces or integrated into a device or applicator such as a toothbrush, dental aligners or roll-ons. Such applicators may be for single or multiple use.

As used herein, the term “dentifrice” includes paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

As used herein, the term “dispenser” refers to any pump, tube, or container suitable for dispensing compositions such as dentifrices.

As stated above the inventors identified a number of compounds or compositions which can be used in oral healthcare compositions: An aspect of the invention provides an oral care composition comprising one or more of the following compounds or compositions:myristic acid, cis-3-hexenylhexanoate, citronellol, nootkatone, ambrette seed absolute, spinach absolute, helichrysum italicum absolute, butanol, geraniol, beta ionone, isoamyl alcohol, methly (2-pentyl-3-oxocyclopentyl) acetate, anethole, decanoic acid, undecanoic acid, nutmeg oil, clary sage oil, lemongrass oil, lime oil, peppermint oil (for example, mint piperita reco nat, mint piperita boulder sx, or mint piperita yakima single cut), gamma octalactone, 3-hexenyl acetate, lavender oil, cinnamaldehyde, clove oil, mint arvensis oil (for example mint arvensis tpc), linseed oil, and thymol.

The compounds or compositions used in the oral care composition of the invention are discussed below.

“Myristic acid” is a material well known in the art. It can be found in nutmeg butter. By “myristic acid” we include material registered under CAS 544-63-8 and is commercially available from many sources.

“cis-3-hexenylhexanoate” is a material well known in the art. It is a fruity green pear-like oror and is used in perfumery. By “cis-3-hexenylhexanoate” we include material registered under CAS 31501-11-8 and is commercially available from many sources.

“Citronellol” is a material well known in the art. It is a fresh floral rosy note and is used in perfumery. By “citronellol” we include material registered under CAS 000106-22-9 and is commercially available from many sources.

“Nookatone” is a material well known in the art. It is an aromatic most well known for its presence in grapefruit. By “nookatone” we include material registered under CAS 004674-50-4 and is commercially available from many sources.

“Ambrette seed absolute” is a material well known in the art. It is used in perfumery and has dry musky aroma. By “ambrette seed absolute” we include material registered under CAS 8015-62-1 and is commercially available from many sources.

“Spinach absolute” is material well known in the art. It is used in perfumery and has a spinach-like aroma profile. By “spinach absolute” we include material registered under CAS 68917-48-6 and is commercially available from many sources.

“Helichrysum italicum absolute” is material well known in the art. It is used in perfumery and has a floral-ambery note. By “helichrysum italicum absolute” we include material registered under CAS 0008023-95-8 and is commercially available from many sources.

“Butanol” is a material well known in the art. It can be used as a base for perfumes, and also has an alcohol aroma. By “butanol” we include material registered under CAS 71-36-3 and is commercially available from many sources.

“Geraniol” is a material well known in the art. It is a common feature of many essential oils, has a rose-like scent and is used in perfumes and flavours. By “geraniol” we include material registered under CAS 106-24-1 and is commercially available from many sources.

“Beta ionone” (which has the chemical formula (3E)-4-(2,6,6-Trimethylcyclohex-1-en-1-yl)but-3-en-2-one), is a well-known material in the art. It is a common feature of many essential oils, has a rose-like scent and is used in perfumes and flavours. By “beta ionone” we include material registered under CAS 79-77-6 and is commercially available from many sources.

“Isoamyl alcohol” is a material well known in the art. It has a fruity, banana like aroma and is used in perfumes and flavours. By “isoamyl alcohol” we include material registered under CAS 123-51-3 and is commercially available from many sources.

“Methyl (2-pentyl-3-oxocyclopentyl) acetate” is a compound used in perfumery. It has a floral, jasmine note with citrus freshness. By “methyl (2-pentyl-3-oxocyclopentyl) acetate” we include material registered under CAS 0024851-98-7 which and is commercially available from Firmenich SA (www.firmenich.com).

“Anethole” is a material well known in the art. It is used in perfumery and has a sweet licorice-like aroma. By “anethole” we include material registered under CAS 004180-23-8 and is commercially available from many sources.

“Decanoic acid” is a material well known in the art. It is used in perfumery and has a sweet licorice-like aroma. By “decanoic acid” we include material registered under CAS 334-48-5 and is commercially available from many sources.

“Undecanoic acid” is a material well known in the art. It is used in perfumery and has a waxy creamy cheese-like aroma. By “undecanoic acid” we include material registered under CAS 112-37-8 and is commercially available from many sources.

“Nutmeg oil” is a material well known in the art. It is used in perfumery and has a spicy, woody aroma. By “nutmeg oil” we include material registered under CAS 8008-45-5 and is commercially available from many sources.

“Clary sage oil” is a material well known in the art. It is used in perfumery and has a fresh herbal tea-like aroma. By “clary sage oil” we include material registered under CAS 8016-63-5.

“Lemongrass oil” is a material well known in the art. It is a complex oil comprising several compounds, including citral, myrcene and limonene, and is prepared from the leaves and woody stalks of the Cymbopogon citratus plant. The oil is commonly used for culinary and health purposes. By “Lemongrass oil” we include material registered under CAS 8007-02-1 which is commercially available from many sources.

“Lime oil” is a material well known in the art. It is a complex oil comprising several compounds, including limonene, γ-terpinene, and β-pinene, and is prepared from the Citrus aurantifolia plant. By “lime oil” we include material registered under CAS 8008-26-2 which and is commercially available from many sources,

“Peppermint oil” is a material well known in the art. It is complex oil comprising several compounds including menthol, menthone, cineol, and is prepared from the leaves and stems of the Mentha×piperita plant. The oil is commonly used for culinary and health purposes. By “Peppermint oil” we include oils obtained from a number of different plant varieties. For example, the peppermint oil known as MINT PIPERITA RECO NAT (material registered under CAS 0068917-18-0), MINT PIPERITA BOULDER SX (material registered under CAS 0008006-90-4) and MINT PIPERITA YAKIMA SINGLE CUT (material registered under CAS 8006-90-4) are included in the scope of “peppermint oil” for the purposes of this application. The “peppermint oil” listed above are commercially available from many sources, for example Essex Laboratories (http://www.essexlabs.com/).

“Gamma octalactone” is a material well known in the art. It has a sweet, coconut odour and is used in the preparation of perfumes and other aroma materials. By “gamma octalactone” we include material registered under CAS 104-50-7 and is commercially available from many sources.

“3-hexenyl acetate” is a material well known in the art. It has a green, fruity odour and is used in the preparation of perfumes and other aroma materials. By “3-hexenyl acetate” we include material registered under CAS 1708-82-3 and is commercially available from many sources.

“Lavender oil” is a material well known in the art. It is complex oil comprising several compounds, including linalool and linalyl acetate, and is prepared from Lavandula species, most commonly Lavandula angustifolia and Lavandula latifolia. Lavender oil has long been used for perfumery purposes. By “lavender oil” we include material registered under CAS 8008-28-0 which and is commercially available from many sources, for example.

“Cinnamaldehyde” is a material well known in the art. It has a cinnamon-like aroma and is used in perfumes and flavours. By “cinnamaldehyde” we include material registered under CAS 14371-10-9 and is commercially available from many sources.

“Clove oil” is a material well known in the art. It is a complex oil comprising several compounds, including eugenol, eugenyl acetate and caryophyllene, and is prepared from the Syzygium aromaticum plant. Clove oil has long been used for perfumery and medicinal purposes. By “clove oil” we include material registered under CAS 8008-34-8 which and is commercially available from many sources.

“Mint arvensis oil” “is a material well known in the art. It is a menthol mint oil, like peppermint, comprising several compounds, including menthol and menthone, and is prepared from the Menthe arvensis plant. Mint arvensis oil has long been used for perfumery and flavour purposes. By “mint arvensis oil” we include material registered under CAS 68917-18-0 which and is commercially available from many sources, for example Sharp Mint Ltd (http://www.sharpmint.com/) and Swati Menthol Allied (http://swatimenthol.com/).

“Linseed oil” “is a material well known in the art. It is complex oil comprising several compounds, including linolenic acid, palmitic acid and stearic acid, and is prepared from the flax plant (Linum usitatissimum). Linseed oil has long been used for industrial purposes. By “linseed oil” we include material registered under CAS 60-33-3 which and is commercially available from many sources. In a preferred embodiment the linseed oil comprises a mixture of the linoleic acid isomers (Z,Z)-9,12-octadecadienoic acid and (Z,Z,Z)-9,12,15-octadecatrienoic acid.

“Thymol” is a material well known in the art. It has been used for perfumery and medicinal purposes. By “thymol” we include material registered under CAS 89-83-8 which and is commercially available from many sources.

Preferably the oral care composition of the invention comprises one or more compounds or compositions which have a positive result in two or more of the assays used in the aspects of the invention described herein. Hence a further embodiment of the invention is where in the oral care composition comprises one or more of the following compounds or compositions: myristic acid, cis-3-hexenylhexanoate, citronellol, nootkatone, ambrette seed absolute, spinach absolute, helichrysum italicum absolute, butanol, geraniol, beta ionone, isoamyl alcohol, methly (2-pentyl-3-oxocyclopentyl) acetate, anethole, decanoic acid, undecanoic acid, nutmeg oil, clary sage oil, lemongrass oil, lime oil, peppermint oil (for example, mint piperita reco nat, mint piperita boulder sx, or mint piperita yakima single cut), gamma octalactone, 3-hexenyl acetate, lavender oil, cinnamaldehyde, clove oil, mint arvensis oil (for example mint arvensis tpc), linseed oil, and thymol.

More preferably the oral care composition of the invention comprises one or more compounds or compositions which have a positive result in all three of the assays used in the aspects of the invention described herein. Hence a further embodiment of the invention is where in the oral care composition comprises one or more of the following compounds or compositions: myristic acid, citronellol, nookatone, nutmeg oil, clary sage oil, lemongrass oil, peppermint oil and gamma octalactone.

As stated above, the oral care composition of the invention comprises one or more of the compounds or compositions described herein. An embodiment of the invention is wherein the oral care composition of the invention comprises two, three, four, five, six, seven, eight, nine, ten, eleven, twelve, thirteen, fourteen, fifteen or more of the following compounds or compositions: myristic acid, cis-3-hexenylhexanoate, citronellol, nootkatone, ambrette seed absolute, spinach absolute, helichrysum italicum absolute, butanol, geraniol, beta ionone, isoamyl alcohol, methly (2-pentyl-3-oxocyclopentyl) acetate, anethole, decanoic acid, undecanoic acid, nutmeg oil, clary sage oil, lemongrass oil, lime oil, peppermint oil (for example, mint piperita reco nat, mint piperita boulder sx, or mint piperita yakima single cut), gamma octalactone, 3-hexenyl acetate, lavender oil, cinnamaldehyde, clove oil, mint arvensis oil (for example mint arvensis tpc), linseed oil, and thymol.

As shown in the accompanying examples, the inventors prepared a series of oral care compositions which comprised combinations of the compounds or compositions described herein. They are termed “flavor compositions A to D”. The series of oral care compositions showed good in vitro and in vivo anti-biofilm activity.

Hence a preferred embodiment is wherein oral care composition of the invention comprises isoamyl alcohol, linseed oil and methyl (2-pentyl-3-oxocyclopentyl). Preferably the oral care composition of the invention comprises 10% isoamyl alcohol, 10% linseed oil and 2.5% methyl (2-pentyl-3-oxocyclopentyl).

A further preferred embodiment is wherein oral care composition of the invention comprises mint arvensis tpc and mint piperita yakima single cut. Preferably the oral care composition of the invention comprises 16.67% mint arvensis tpc and 16.67% mint piperita yakima single cut.

A still further preferred embodiment is wherein oral care composition of the invention comprises cinnamaldehyde and clove oil. Preferably the oral care composition of the invention comprises 15% cinnamaldehyde and 15% clove oil.

As can be appreciated the amount of the compounds or composition can vary according to the specific compounds or composition and the formation and intended use.

However, an embodiment of the invention provides an oral care composition of the invention is wherein the compounds or compositions is at a concentration of from 0.001 to 5%, particularly preferably from 0.005 to 3%, preferably 0.5 to 2% in each case with respect to the total weight of the composition.

An embodiment of the invention provides an oral care composition of the invention, wherein said composition further comprising one or more of the following ingredients: flavor system, cooling agents, surfactant, humectant, pigments, antibacterial agents, thickening agents, fluoride sources, tartar control agents and/or preservatives.

In some aspects, the composition further comprises a flavor system. The flavor system may mask any unpleasant taste and sensations due to certain components of the composition such as antimicrobial actives or peroxide. Without intending to be limited to any particular theory, pleasant tasting compositions improve user compliance to prescribed or recommended use of oral care products. The flavor system may also comprise traditional flavor components, in particular those that are relatively stable in the presence of usual oral care product carrier materials or excipients. The combination of the selected flavoring system with the compositions presented herein may provide a high-impact refreshing sensation with a well-rounded flavor profile.

In some aspects, the flavor system may comprise additional flavor ingredients including but not limited to, oil of wintergreen, cassia, sage, parsley oil, marjoram, orange, cis-jasmone, 2,5-dimethyl-4-hydroxy-3(2H)-furanone, 5-ethyl-3-hydroxy-4-methyl-2(5H)-furanone, vanillin, ethyl vanillin, anisaldehyde, 3,4-methylenedioxybenzaldehyde, 3,4-dimethoxybenzaldehyde, 4-hydroxybenzaldehyde, 2-methoxybenzaldehyde, benzaldehyde; propenyl guaethol, heliotropine, 4-cis-heptenal, diacetyl, methyl-p-tert-butyl phenyl acetate, methyl salicylate, ethyl salicylate, 1-menthyl acetate, oxanone, alpha-irisone, ethyl butyrate, ethyl acetate, methyl anthranilate, iso-amyl acetate, iso-amyl butyrate, allyl caproate, eugenol, eucalyptol, octanol, octanal, decanol, decanal, phenylethyl alcohol, benzyl alcohol, alpha-terpineol, maltol, ethyl maltol, anethole, dihydroanethole, carvone, menthone, b-damascenone, and mixtures thereof.

Generally suitable flavoring ingredients are those containing structural features and functional groups that are less prone to redox reactions. These include derivatives of flavor chemicals that are saturated or contain stable aromatic rings or ester groups. Also suitable are flavor chemicals that may undergo some oxidation or degradation without resulting in a significant change in the flavor character or profile. The flavor ingredients may be supplied in the composition as single or purified chemicals or by addition of natural oils or extracts that have preferably undergone a refining treatment to remove components that are relatively unstable and may degrade and alter the desired flavor profile, resulting in a less acceptable product from an organoleptic standpoint. Flavoring agents may generally be used in the compositions at levels of from about 0.001% to about 5%, by weight of the composition.

In some aspects, the flavor system may further include a sweetening agent. Suitable sweeteners include those well known in the art, including both natural and artificial sweeteners. Some suitable water-soluble sweeteners include monosaccharides, disaccharides and polysaccharides such as xylose, xylitol, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin. Also included are monk fruit derived sweeners, including mogrosides. Also included are sweetners derived from Stevia rebaudiana including stevioside and rebaudioside (particularly Rebaudioside A). Suitable water-soluble artificial sweeteners include soluble saccharin salts, i.e., sodium or calcium saccharin salts, cyclamate salts, the sodium, ammonium or calcium salt of 3, 4-dihydro-6-methyl-1, 2, 3-oxathiazine-4-one-2, 2-dioxide, the potassium salt of 3, 4-dihydro-6-methyl-1, 2, 3-oxathiazine-4-one-2, 2-dioxide (acesulfame-K), the free acid form of saccharin, and the like. Other suitable sweeteners include dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2, 5, dihydrophenyl-glycine, L-aspartyl-2,5-dihydro-L-phenylalanine, L-aspartyl-L-(I-cyclohexylen)-alanine, and the like. Water-soluble sweeteners derived from naturally occurring water-soluble sweeteners, such as a chlorinated derivative of ordinary sugar (sucrose), known, for example, under the product description of sucralose as well as protein based sweeteners such as Thaumatoccous daniellii (Thaumatin I and II) can be used.

In some aspects, the composition may contains from about 0.1% to about 10% of sweetener, alternatively from about 0.1% to about 1%, by weight of the composition.

In some aspects, the flavor system may further include salivating agents, warming agents, and numbing agents. These agents may be present in the compositions at a level of from about 0.001% to about 10%, alternatively from about 0.1% to about 1%, by weight of the composition.

Suitable salivating agents include Jambu® manufactured by Takasago. Suitable numbing agents include benzocaine, lidocaine, clove bud oil, and ethanol. Examples of warming agents include ethanol, capsicum and nicotinate esters, such as benzyl nicotinate.

In some aspects, the oral care composition of the invention may further include cooling agents such as those set forth in U.S. Pat. Nos. 9,394,287 and 9,732,071. Additional examples of cooling agents include 5-methyl-2-(propane-2-yl)cyclohexyl-N-ethyloxamate, N-ethyl-p-menthane carboxamide (WS-3, also referred to as menthane-3-carboxylic acid-N-ethyl amide), N-2,3-trimethyl-2-isopropyl butane amide (WS-23), menthyl lactate (Frescolat® ML), menthone glycerine acetal (Frescolat® MGA), mono-menthyl succinate (Physcool®), mono-menthyl glutarate, O-menthyl-glycerine, menthyl-N,N-dimethyl succinamate, N-(4-cyano methyl phenyl)-p-menthane carboxamide, N-(2-(pyridin-2-yl)ethyl)-3-p-menthane carboxamide, menthol and menthol derivatives (e.g. L-menthol, D-menthol, racemic menthol, isomenthol, neoisomenthol, neomenthol) menthyl ether (e.g. (1-menthoxy)-1,2-propanediol, (I-menthoxy)-2-methyl-1,2-propanediol, 1-menthyl-methyl ether), menthyl ester (e.g. menthyl formiate, menthyl acetate, menthyl isobutyrate, menthyl lactates, L-menthyl-L-lactate, L-menthyl-D-lactate, menthyl-(2-methoxy)acetate, menthyl-(2-methoxy ethoxy)acetate, menthyl pyroglutamate), N-(4-cyano methyl phenyl)-p-menthane carboxamide, N-(2-(pyridin-2-yl)ethyl)-3-p-menthane carboxamides, menthyl carbonates (e.g. menthyl propylene glycol carbonate, menthyl ethylene glycol carbonate, menthyl glycerine carbonate or mixtures thereof), menthane carboxylic acid amide (e.g. menthane carboxylic acid-N-ethylamid [WS3], N-alpha.-(menthane-carbonyl)glycine ethyl ester [WS5], menthane carboxylic acid-N-(4-cyanophenyl)amide, menthane carboxylic acid-N-(alkoxyalkyl)amide), menthone and menthone derivatives (e.g. L-menthone glycerine ketal), 2,3-dimethyl-2-(2-propyl)-butyric acid derivatives (e.g. 2,3-dimethyl-2-(2-propyl)-butyric acid-N-methyl amide [WS23]), isopulegol or its esters (1-(−)-isopulegol, 1-(−)-isopulegol acetate), menthane derivatives (e.g. p-menthane-3,8-diol), N-(4-cyano methyl phenyl)-p-menthane carboxamides, N-(2-(pyridin-2-yl)ethyl)-3-p-menthane carboxamides, cubebol or synthetic or natural mixtures containing cubebol, pyrrolidone derivates of cycloalkyl dione derivatives (e.g. 3-methyl-2(1-pyrrolidinyl)-2-cyclopentene-1-one) or tetrahydropyrimidine-2-ones (e.g. Icilin or related compounds such as those described in WO 2004/026840), N-(4-cyano methyl phenyl)-p-menthane carboxamide, N-(2-(pyridin-2-yl)ethyl)-3-p-menthane carboxamides, menthyl ether (e.g. (1-menthoxy)-1,2-propanediol, (1-menthoxy)-2-methyl-1,2-propanediol), more polar menthyl esters (e.g. menthyl lactates, L-menthyl-L-lactate, L-menthyl-D-lactate, menthyl-(2-methoxy)acetate, menthyl-(2-methoxy ethoxy)acetate, menthyl pyroglutamate), menthyl carbonates (e.g. menthyl propylene glycol carbonate, menthyl ethylene glycol carbonate, menthyl glycerine carbonate), the semi-esters of menthols with a dicarboxylic acid or the derivatives thereof (e.g. mono-menthyl succinate, mono-menthyl glutarate, mono-menthyl malonate, O-menthyl succinic acid ester-N,N-(dimethyl)amide, O-menthyl succinic acid ester amide), 3,4-methylendioxycinnamic acid-N-cyclohexyl-N-2-pyridylamide, isopropyl-(5-methoxy-2-pyridin-2-yl-pyrimidin-4-yl)-amine, 3,4,6,7,11 b,12-hexahydro-3,3-dimethyl-spiro[13H-dibenzo[a,f]quinolizine-1-3,2′-[1,3]dithiolan]-1(2H)-one, 5,6,10b,1 1-tetrahydro-3-methyl-spiro[12H-benzo[a]furo[3,4-f]quinolizine-1-2,2′-[1,3]dithiolan]-1(3H)-one. Most preferred as cooling compounds are compounds selected from the group consisting of 5-methyl-2-(propane-2-yl)cyclohexyl-N-ethyloxamate, N-ethyl-p-menthane carboxamide (WS-3, also referred to as menthane-3-carboxylic acid-N-ethyl amide), menthyl lactate (Frescolat® ML), menthone glycerine acetal (Frescolat® MGA), N-(4-cyano methyl phenyl)-p-menthane carboxamide and (I-menthoxy)-1,2-propanediol, 2-(4-methylphenoxy)-N-(1H-pyrazol-5-yl)-N-(2-thienylmethyl)acetamide, 2-(4-methylphenoxy)-N-(1H-pyrazol-3-yl)-N-(2-thienylmethyl)acetamide and a mixture of 2-(4-methylphenoxy)-N-(1H-pyrazol-5-yl)-N-(2-thienylmethyl)acetamide and 2-(4-methylphenoxy)-N-(1H-pyrazol-3-yl)-N-(2-thienylmethyl)acetamide.

In addition to the components described above, the present compositions may comprise additional optional components and/or orally acceptable carrier materials.

In some aspects, the oral care composition comprises a surfactant component.

Any orally acceptable surfactant, most of which are anionic, nonionic or amphoteric, can be used. Suitable anionic surfactants include without limitation, sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable nonionic surfactants include poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants include derivatives of Cs-2o aliphatic secondary and tertiary amines having an anionic group such as carboxylate, sulfate, sulfonate, phosphate or phosphonate.

In some embodiments the surfactant component comprises (i) a surfactant selected from the group consisting of: poly(oxyethylene)-poly(oxypropylene)-poly(oxyethylene) block copolymers, poly(oxyethylene)-modified hydrogenated castor oils, and poly(oxyethylene)-modified fatty acid monoesters of sorbitan, and (ii) a surfactant selected from the group consisting of polyol esters and sugar esters.

In some aspects, the oral care composition comprises a pigment. Suitable orally acceptable can be used, including talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride.

In some aspects, the oral care composition comprises a fluoride ion source. Suitable fluoride ion sources include stannous fluoride, sodium fluoride, potassium fluoride, potassium monofluorophosphate, sodium monofluorophosphate, ammonium monofluorophosphate, sodium fluoro silicate, ammonium fluorosihcate, an amine fluoride, ammonium fluoride, and combinations thereof.

In some aspects, the oral care composition comprises antibacterial agents. Suitable antibacterial agents include ethanol, triclosan, cetylpyridinium chloride (CPC) Chlorhexidine (CHX), Isopropyl Methylphenol (IPMP), Isopropyl Methylphenol (IPMP), Zinc sulfate (ZnSO4), Zinc Citrate, Zinc chloride (ZnCl2).

An embodiment of the invention provides an oral care composition of the invention is wherein the composition is coated or encapsulated.

Various methods are known in the art for preparing oral care compositions in which one or more components are coated or encapsulated in a powder-coated dissolvable film fragments, e.g., wherein the film matrix comprises a water-insoluble active agent.

A further aspect of the invention provides an oral care product or product for nutrition or pleasure comprising an oral care composition of the invention.

In some aspects, the oral care composition comprises a humectant component. Examples of humectants include: Propylene glycol, hexylene glycol, and butylene glycol, Aloe vera gel, Alpha hydroxy acids such as lactic acid, Egg yolk and egg white, Glyceryl triacetate, Honey, Lithium chloride, Molasses, Polymeric polyols such as polydextrose, Quillaia, Sodium hexametaphosphate E452i, Sugar alcohols (sugar polyols) such as glycerol, sorbitol, xylitol, maltitol, Urea and Castor

A further aspect of the invention provides one or more of the following compounds or compositions: lemongrass oil, lime oil, peppermint oil (for example, mint piperita reco nat, mint piperita boulder sx, or mint piperita yakima single cut), gamma octalactone, 3-hexenyl acetate, butanol, geraniol, lavender oil, beta ionone, isoamyl alcohol, cinnamaldehyde, clove oil, mint arvensis oil (for example mint arvensis tpc), linseed oil, methyl (2-pentyl-3-oxocyclopentyl) acetate and thymol, or an oral care composition of any of the previous aspects of the invention, for use as anti-oral biofilm agent.

A further aspect of the invention provides a consumer food or beverage product comprising an oral care composition of the invention.

A preferred embodiment of the consumer food or beverage product wherein the oral care composition of the invention comprises isoamyl alcohol, linseed oil and methyl (2-pentyl-3-oxocyclopentyl). Preferably the oral care composition of the invention comprises 10% isoamyl alcohol, 10% linseed oil and 2.5% methyl (2-pentyl-3-oxocyclopentyl).

A preferred embodiment of the consumer food or beverage product wherein the oral care composition of the invention comprises mint arvensis tpc and mint piperita yakima single cut. Preferably the oral care composition of the invention comprises 16.67% mint arvensis tpc and 16.67% mint piperita yakima single cut.

A preferred embodiment of the consumer food or beverage product wherein the oral care composition of the invention comprises cinnamaldehyde and clove oil. Preferably the oral care composition of the invention comprises 15% cinnamaldehyde and 15% clove oil. By “consumer food or beverage product” we include, by way of example, confections such as chewing gum, lozenges, hard candies, beverages such as soft drinks and flavored water, dairy beverages and products such as milk preparations, yogurt drinks, lactose-free yogurt, acidified milk, coffee cream or whipped cream, cheese, quark, ice cream, and other frozen desserts. Also included are alcoholic and non-alcoholic beverages. Other suitable food and beverage products would be well known to the skilled person.

A further aspect of the invention provides a method of treatment or prevention of erosive tooth demineralization, gingivitis, plaque, periodontitis, halitosis and/or dental caries, the method comprising the application to the oral cavity of a person in need thereof a composition according to any preceding aspect of the invention.

A further aspect of the invention provides use of an oral care composition according to any aspect of the invention for treatment or prevention of erosive tooth demineralization, gingivitis, plaque, periodontitis, halitosis and/or dental caries.

By “anti-oral biofilm agent” we include where the compounds or compositions prevent or treat oral pathologies associated with oral biofilms. Hence the invention includes where the compounds and compositions specified herein are for use in preventing or treating erosive tooth demineralization, gingivitis, plaque, periodontitis, halitosis and/or dental caries.

A further aspect of the invention provides a method of identifying a test composition having use in oral healthcare comprising
(a) assaying whether the test composition reduces bacteria adhesion, and/or,
(b) assaying whether the test composition reduces biofilm formation, and/or,
(c) assaying whether the test composition disrupts the biofilm,
wherein a composition which is positive in one or more assays is of use in an oral healthcare composition.

The method of the invention determines whether a test composition has use in oral healthcare according to a positive result in one or more of the assays used in the aspect of the invention. By “positive result” the inventors mean that the composition gives a statistically significant effect in the assay compared to a control reaction which includes the same sample mixture but lacking the test composition.

The accompanying examples provides details of the method of each assay of this method of the invention. Further information is also provided below.

One assay is used to examine whether the test composition reduces bacteria adhesion. When performing this assay, a tooth enamel substitute material is used as a substrate which is suitable for the adhesion of oral biofilm generating bacteria. The tooth enamel substitute material is exposed to oral biofilm generating bacteria with and without the presence of the test composition, and after a suitable incubation time the effect of the test composition on bacteria adhesion is determined. Hence a reduction in oral biofilm generating bacteria adhesion to a tooth enamel substitute material is beneficial since it reduces the formation of biofilm on the tooth and therefore reduces pathologies associated with oral biofilms (as described above).

Therefore the assay is designed to mimic the attachment of initial colonizing species of bacterial onto a sterile surface, specifically a tooth enamel substitute material. The assay is performed with and without treatment with a test composition. This is step (a) of the assays listed in the method of the invention.

Hence an embodiment of the method of identifying a test composition is wherein the assay of step (a) comprises exposing a substrate to saliva and/or bacteria in the addition or absence of a test composition and determining whether the test composition reduces bacteria adhesion to the substrate.

Suitable tooth enamel substitute materials include Hydroxyappatite (HA) disks or bovine dentin and enamel. The tooth enamel substitute materials are exposed to the oral biofilm generating microorganisms with and without the presence of the test composition for a certain period of time, for example 30 mins to 24 hours, preferably 24 hours. After this the biofilm is harvested and analysed by recording the number and state of the microorganisms.

A further assay is used to examine whether the test composition reduces biofilm formation. This assay mimics the development of dental biofilms at early stages. When performing this assay, assay plates with saliva coated wells are exposed to test compositions for a number of times during an incubation period, after which the effect of the test composition on biofilm formation is analysed by recording the number and state of the microorganisms.

The assay plates are exposed to test composition for a number of times, preferably twice within a 24 hour period.

Therefore the assay is designed to mimic the early stage when biofilm is being formed. The assay is performed with and without treatment with a test composition, and the treatment is applied as the biofilm develops on a tooth enamel substitute material, as explained above. This is step (b) of the assays listed in the method of the invention.

Hence embodiment of the method the invention is wherein the assay of step (b) comprises allowing exposing a substrate to saliva and/or bacteria under culture conditions for at least 24 hours then exposing the cultured mixture to a test composition and determining whether the test composition reduces biofilm formation to the substrate.

A further assay is used to examine whether the test composition disrupts the biofilm. This assay is designed to evaluate the ability of a test composition to disrupt mature biofilms. A biofilm is prepared according to the formation assay as described above. Then, when the biofilm is mature (approximately 3 to 7 days after the start of incubation), it is exposed to the test composition for a number of times during a test period, after which the time the effect of the test composition on biofilm formation is analysed by recording the number and state of the microorganisms.

Therefore the assay is designed to mimic the mature biofilms. The assay is performed with and without treatment with a test composition, and the treatment is applied after the biofilm has developed on a tooth enamel substitute material, as explained above, for at least 3 days, preferably around seven days. This is step (c) of the assays listed in the method of the invention.

An embodiment of the method of identifying a test composition is wherein the assay of step (c) comprises allowing a biofilm to form on a substrate by exposing a substrate to saliva and/or bacteria under culture conditions for at least 72 hours then exposing the cultured mixture to a test composition and determining whether the test composition disrupts the biofilm.

A further embodiment of the method of identifying a test composition is wherein the effect of the test composition on the biofilm is determined by harvesting the biofilm and determining the amount and type of bacteria present in the biofilm.

A further embodiment of the method of identifying a test composition comprises preparing an oral care composition using a test composition which is positive in one or more assays.

The invention will now be described in further detail by way of the following examples which illustrate the benefits and advantages of the present invention.

### EXAMPLES

### Example 1. In Vitro Screening for Anti-Biofilm Activities of Flavor Ingredients

### Inocula Preparation.

A total of 12 bacterial strains were selected based on scientific literature. The strains were grown in media recommended by strain suppliers. Broth cultures were adjusted to log 7 CFU/mL with growth media containing 20% of glycerol. For C. matruchotii growth medium with glycerol was used to wash off the colonies culture plates, the cell suspension was then homogenized by vortexing with glass beads. The aerobic and anaerobic strains were mixed and aliquoted separately. The inocula were stored at −80° C. until use.

TABLE 1
Bacterial strains used as inocula

| Atmosphere | Strain |
|---|---|
| Anaerobic | P. melaninogenica ATCC 25845 |
| | F. nucleatum DSM 20482 |
| | V. parvula DSM 2007 |
| | C. ochracea DSM 7271 |
| 5% CO2 | S. oralis DSM 20627 |
| | S. mitis NCIMB 13770 |
| | S. mutans ATCC 25175 |
| | S. sanguinis DSM 20068 |
| | A. naeslundii ATCC 19039 |
| | A. odontolyticus ATCC 17982 |
| | A. oris DSM 23056 |
| | C. matruchotii DSM 20635 |

Saliva and oral inocula preparation. Saliva and oral microflora samples were collected from healthy volunteers by passive drooling using a method described previously [1].
(a) Bacteria Adhesion assay. Pegs of 96-well MBEC plate (Innovotech, Canada) were submerged in saliva for pellicle formation. Overnight culture of A. oris, A. naeslundii, S. mitis and S. oralis were normalized to OD=0.1 using 0.85% NaCl and mixed in equal proportions. The plates were treated with samples for 2 min and incubated with the culture mixture for 30 min at 37° C. and 5% CO2. The pegs on 96-well plates were then washed 3 times before harvest.
(b) Biofilm formation assay. Saliva-coated 96-well MBEC plates were incubated with inocula anaerobically overnight. The next day, the inoculum was replaced with modified BHI broth (BHI supplemented with 1 g/L mucin, 0.5 mg/L Vitamin K and 10 mg/L hemin). The devices were treated for 2 min twice to mimic the application of dental care products, after which the treatment solution was replaced with fresh modified BHI broth and incubation of the devices was continued. The biofilms were harvested after two days.
(c) Biofilm disruption assay. The inoculation and incubation was done in the same manner as described in the formation assay except that the incubation time was extended to 7 days, after which the mature biofilm was subjected to treatment twice a day as in the formation assay for 3 days before harvest.

Biofilm harvest. To harvest the attached cells, the peg of the MBEC device was immersed in 200 μL of saline solution per well. The plates were sonicated in an ultrasonic bath to obtain cell suspension. For the adhesion assay, the suspension was serially diluted and plated on Tryptic Soy Agar using Microlab STAR (Hamilton). For the formation and disruption assays, the suspension was used for SYTO 9 staining using LIVE/DEAD® BacLight Bacterial Viability Kits L7012 (Thermo Fisher Scientific) following manufacturer instructions.

### Results

Table 2 lists all the compounds and compositions that resulted in significant biofilm reduction in any of the three assays.

TABLE 2
Compounds and compositions tested positive in anti-biofilm assays using defined
inocula. p-value, statistical significance of t-test between control base and
treatments; Δ, difference in relative biofilm abundance between control base
and treatments. For adhesion assay, the biofilms were quantified as relative CFU
with base designated as 100. For formation and disruption assays, the biofilms
were quantified as relative fluorescence intensity with base designated as 100.

| | | In vitro antibiofilm effect | | | | | |
|---|---|---|---|---|---|---|---|
| | | Adhesion | | Formation | | Disruption | |
| Material | Dosage | p-value | Δ | p-value | Δ | p-value | Δ |
| LIME OIL | 0.1% | 0.05 | 79.4 | 0.046 | 83.5 | 0.003 | 56.3 |
| LEMONGRASS OIL | 0.01% | 0.04 | 42.4 | 0.437 | 29.3 | 0.090 | 40.7 |
| MINT PIPERITA RECO | 0.10% | 0.04 | 75.4 | 0.965 | 1.7 | 0.161 | 37.5 |
| NAT | | | | | | | |
| OCTALACTONE | 0.01% | 0.01 | 63.5 | 0.003 | 89.1 | 0.016 | 74.1 |
| 3-HEXENYL ACETATE | 0.01% | 0.24 | 32.7 | 0.001 | 78.1 | 0.022 | 64.7 |
| BUTANOL | 0.10% | 0.11 | 56.8 | 0.032 | 83.5 | 0.002 | 59.5 |
| GERANIOL | 0.01% | 0.08 | 32.1 | 0.001 | 84.7 | 0.035 | 61.7 |
| LAVENDER OIL | 0.01% | 0.19 | 24.6 | 0.019 | 62.4 | 0.050 | 52.3 |
| MINT PIPERITA | 0.10% | 0.05 | 76.0 | 0.046 | 78.2 | 0.004 | 51.7 |
| BOULDER SX | | | | | | | |
| BETA IONONE | 0.01% | 0.21 | 19.4 | 0.001 | 80.8 | 0.030 | 61.8 |
| ISOAMYL ALCOHOL | 0.10% | 0.06 | 63.7 | 0.040 | 82.3 | 0.002 | 57.4 |
| CINNAMALDEHYDE | 0.10% | 0.73 | −9.1 | 0.036 | 83.3 | 0.017 | 48.2 |
| CLOVE OIL | 0.01% | 0.43 | 14.7 | 0.001 | 80.7 | 0.017 | 70.9 |
| MINT ARVENSIS TPC | 0.10% | 0.08 | 58.5 | 0.042 | 85.6 | 0.003 | 53.1 |
| MINT PIPERITA YAKIMA | 0.10% | 0.79 | 9.3 | 0.043 | 79.1 | 0.009 | 48.6 |
| SINGLE CUT | | | | | | | |
| LINSEED OIL | 0.10% | 0.35 | −23.8 | 0.049 | 76.8 | 0.003 | 62.2 |
| METHLY (2-PENTYL-3- | 0.01% | 0.06 | 37.1 | 0.006 | 86.3 | 0.033 | 63.2 |
| OXOCYCLOPENTYL) | | | | | | | |
| ACETATE | | | | | | | |
| THYMOL | 0.10% | 0.85 | 5.7 | 0.005 | 86.7 | 0.027 | 72.0 |

### Example 2. In Vitro Experiment for Anti-Biofilm Activities of Compounds and Compositions Using Oral Inocula

Flavor compositions containing the compounds and compositions listed in Table 2 were assessed for their antibiofilm activities with biofilms derived from oral samples taken from volunteers. Table 3 lists the ones that resulted in significant biofilm reduction in any of the three assays.

The flavor compositions used were as follows (all amounts in percentage of overall composition:
Flavor A—10% isoamyl alcohol, 10% linseed oil and 2.5% methyl (2-pentyl-3-oxocyclopentyl)
Flavor B—16.67% MINT ARVENSIS TPC and 16.67% MINT PIPERITA YAKIMA SINGLE CUT
Flavor C—25% MINT PIPERITA YAKIMA SINGLE CUT
Flavor D—15% cinnamaldehyde and 15% clove oil.

Table 3. Flavor compositions tested positive in anti-biofilm assays using oral inocula. p-value, statistical significance of t-test between control base and treatments; A, difference in relative biofilm abundance between control base and treatments. For adhesion assay, the biofilms were quantified as relative CFU with base designated as 100. For formation and disruption assays, the biofilms were quantified as relative fluorescence intensity with base designated as 100.

TABLE 3
Flavor compositions tested positive in anti-biofilm assays using oral
inocula. p-value, statistical significance of t-test between control
base and treatments; Δ, difference in relative biofilm abundance
between control base and treatments. For adhesion assay, the biofilms
were quantified as relative CFU with base designated as 100. For
formation and disruption assays, the biofilms were quantified as
relative fluorescence intensity with base designated as 100.

| | Adhesion | | Formation | | Disruption | |
|---|---|---|---|---|---|---|
| | p-value | Δ | p-value | Δ | p-value | Δ |
| Flavor A | 0.21 | −13.4 | 0.03 | 52.3 | 0.77 | −34.4 |
| Flavor B | 0.31 | −17.6 | 0.24 | 20.5 | 0.00 | 75.1 |
| Flavor C | 0.00 | −63.1 | 0.35 | 32.2 | 0.03 | 77.1 |
| Flavor D | 0.23 | −16.2 | 0.00 | 51.2 | 0.03 | 55.4 |

### Example 3. Composition and Preparation of the End-Product

Table 4. Composition of mouth rinse

Sample mouthwashes were prepared for each of the compounds and compositions listed in Table 2 above and for flavor mixes A to D as specified in Example 2.

TABLE 4

| Ingredients | Amount (% wt) |
|---|---|
| Compounds and compositions in Table 2 | 0.20% |
| or flavor mix specified in Example 2 | |
| RH40 | 0.50% |
| Sodium Saccharin | 0.02% |
| Glycerine | 12.00% |
| Sodium Benzoate | 0.10% |
| DI/Purified Water | Q.S to Final Wt. |

The compounds and compositions in Table 2 or flavor mixes specified in Example 2 were mixed with RH40 (Component A). The rest of the ingredients were mixed to make a master mix (Component B) and then aliquoted. Component A was slowly added to Component B with constant mixing until it is fully dissolved.

### Example 4. The Effect of Mouthwash on Plaque Regrowth In Vivo

The mouth wash solutions prepared in Example 3 were then used in the following experiments.

Volunteers without major oral diseases were recruited. As a measure of oral hygiene status, simplified plaque index, soft debris index and calculus index were assessed by two dentists for 4 times throughout the experiment. On Day 0 of the experiment, the subjects received a professional dental cleansing to remove all calculus and plaque. They were then asked to use 20 mL of mouthwash after the cleaning and refrain from using other dental cleansing products until the next dental check on Day 1. After that, they were instructed to use a standard toothpaste (Lion Floral Mint) to brush twice a day and use mouthwash three times a day. On day 7 and 14, the used mouthwash at noon was collected and the bacterial load was determined by plating on Tryptic Soy Agar and Mitis Salivarius Agar.

### Results

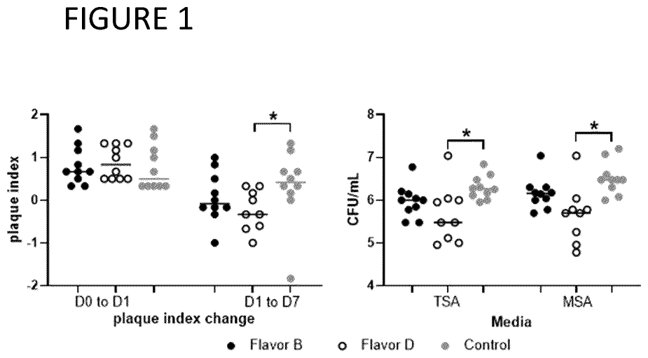

Between Day 7 and Day 1, the increase of average plaque index was significantly lower for the Flavor D treatment group than the control. Meanwhile the bacterial load in the used mouthwash was also lower, indicating that the mouthwash improved oral hygiene of the subjects (FIG. 1).

### Example 5. Further In Vitro Screening for Anti-Biofilm Activities of Flavor Ingredients

The inventors used the assays described above to screen more ingredients for activity in a model mouth wash (Table 5).

The compounds and compositions for testing activities in Table 6 were dissolved in DMSO. The rest of the ingredients of mouth rinse in Table 5 were mixed to make a master mix in water and then aliquoted. The DMSO solution was then added to the master mix and homogenized.

TABLE 5
Composition of mouth rinse
Sample mouthwashes composition for highlighted ingredients

| Ingredients | Amount (% wt) |
|---|---|
| Compounds in Table 6 | 10 mM |
| DMSO | 10% |
| Sodium Saccharin | 0.02% |
| Glycerine | 12.00% |
| Sodium Benzoate | 0.10% |
| DI/Purified Water | Q.S to Final Wt. |

Table 6. Compounds and compositions tested positive in anti-biofilm assays using defined inocula. p-value, statistical significance of t-test between control base and treatments; Δ, difference in relative biofilm abundance between control base and treatments. For adhesion assay, the biofilms were quantified as relative CFU with base designated as 100. For formation and disruption assays, the biofilms were quantified as relative fluorescence intensity with base designated as 100.

TABLE 6

| | | | Adhesion | | Formation | | Disruption | |
|---|---|---|---|---|---|---|---|---|
| | | | p- | | p- | | p- | |
| CAS | name | dosage | value | Δ | value | Δ | value | Δ |
| 112-37-8 | undecanoic acid | 10 mM | 0.010 | 98.8 | 0.000 | 87.5 | 0.102 | 89.1 |
| 000106-22-9 | Citronellol | 10 mM | 0.021 | 91.7 | 0.026 | 58.1 | 0.043 | 67.0 |
| 004674-50-4 | Nootkatone | 10 mM | 0.021 | 86.4 | 0.039 | 20.3 | 0.044 | 63.1 |
| 544-63-8 | Myristic Acid | 10 mM | 0.024 | 33.3 | 0.005 | 64.9 | 0.018 | 87.9 |
| 334-48-5 | Decanoic acid | 10 mM | 0.143 | −420.2 | 0.030 | 93.3 | 0.090 | 89.8 |
| 004180-23-8 | Anethole | 10 mM | 0.678 | 17.4 | 0.037 | 53.4 | 0.010 | 77.4 |
| 31501-11-8 | cis-3-hexenylhexanoate | 10 mM | 0.839 | 9.1 | 0.030 | 58.1 | 0.013 | 75.3 |
| 8016-63-5 | clary sage oil | 10 mM | 0.000 | 81.4 | 0.007 | 68.8 | 0.019 | 86.5 |
| 8008-45-5 | nutmeg oil | 10 mM | 0.001 | 76.9 | 0.006 | 62.5 | 0.018 | 87.0 |
| 68917-48-6 | spinach absolute | 10 mM | 0.002 | 93.9 | 0.178 | 93.3 | 0.810 | −12.5 |
| 8015-62-1 | ambrette seed absolute | 10 mM | 0.001 | −270.6 | 0.014 | 43.1 | 0.007 | 65.1 |
| 0008023-95-8 | helichrysum italicum absolute | 10 mM | 0.891 | −7.7 | 0.025 | 94.4 | 0.033 | 65.0 |

1. An oral care composition comprising one or more of the compounds or compositions selected from the group consisting of myristic acid, cis-3-hexenylhexanoate, citronellol, nootkatone, ambrette seed absolute, spinach absolute, helichrysum italicum absolute, butanol, geraniol, beta ionone, isoamyl alcohol, methyl (2-pentyl-3-oxocyclopentyl) acetate, anethole, decanoic acid, undecanoic acid, nutmeg oil, clary sage oil, lemongrass oil, lime oil, peppermint oil, piperita reco nat, mint piperita boulder sx, mint piperita yakima single cut, gamma octalactone, 3-hexenyl acetate, lavender oil, cinnamaldehyde, clove oil, mint arvensis oil, mint arvensis tpc, linseed oil, and thymol.

2. The oral care composition of claim 1, wherein the one or more of the compounds or compositions is selected from the group consisting of myristic acid, citronellol, nookatone, nutmeg oil, clary sage oil, lemongrass oil, peppermint oil, and gamma octalactone.

3. The oral care composition of any claim 1, wherein the one or more of the compounds or compositions is at a concentration of from 0.001 to 5% with respect to the total weight of the composition.

4. The oral care composition of claim 1, further comprising one or more ingredients selected from the group consisting of flavor systems, cooling agents, surfactants, humectants, pigments, antibacterial agents, thickening agents, fluoride sources, tartar control agents, and preservatives.

5. The oral care composition according to claim 1, wherein the composition is coated or encapsulated.

6. An oral care product or product for nutrition or pleasure comprising an oral care composition according to claim 1.

7. The oral care product of claim 6s wherein the oral care product is selected from the group consisting of tooth paste, tooth powder, tooth gel, tooth cleaning liquid, tooth cleaning foam, tooth tablets, mouth wash, mouth rinse, mouth spray, dental floss, chewing gum, dental aligners, lozenges, dentures, and effervescent tablets.

8. A method of treatment or prevention of erosive tooth demineralization, gingivitis, plaque, periodontitis, halitosis and/or dental caries, the method comprising applying to the oral cavity of a person in need thereof the composition according to claim 1.

9. A method of identifying a test composition having use in oral healthcare, the method comprising
(a) assaying whether the test composition reduces bacteria adhesion, and/or,
(b) assaying whether the test composition reduces biofilm formation, and/or,
(c) assaying whether the test composition disrupts the biofilm,
wherein a composition which is positive in one or more assays is of use in an oral healthcare composition.

10. The method of claim 9, wherein the assay of step (a) comprises exposing a substrate to saliva and/or bacteria in the addition or absence of a test composition and determining whether the test composition reduces bacteria adhesion to the substrate.

11. The method of claim 9, wherein the assay of step (b) comprises allowing exposing a substrate to saliva and/or bacteria under culture conditions for at least 24 hours then exposing the cultured mixture to a test composition and determining whether the test composition reduces biofilm formation to the substrate.

12. The method of claim 9, wherein the assay of step (c) comprises allowing a biofilm to form on a substrate by exposing a substrate to saliva and/or bacteria under culture conditions for at least 72 hours then exposing the cultured mixture to a test composition and determining whether the test composition disrupts the biofilm.

13. The method of claim 9, wherein the effect of the test composition on the biofilm is determined by harvesting the biofilm and determining the amount and type of bacteria present in the biofilm.

14. The method of claim 9, further comprising preparing an oral care composition using a test composition which is positive in one or more assays.

15. The oral care composition of claim 1, wherein the one or more of the compounds or compositions is at a concentration of from 0.005 to 3% with respect to the total weight of the composition.

16. The oral care composition of claim 1, wherein the one or more of the compounds or compositions is at a concentration of from 0.05 to 2.5% with respect to the total weight of the composition.

17. The oral care composition of claim 1, wherein the one or more of the compounds or compositions is at a concentration of from 0.1 to 2.5% with respect to the total weight of the composition.

18. The oral care composition of claim 1, wherein the one or more of the compounds or compositions is at a concentration of from 0.5 to 2% with respect to the total weight of the composition.
